(19) United States
(12) Patent Application Publication
(10) Pub. No.: US 2007/0088361 A1
(43) Pub. Date: Apr. 19, 2007

(54) Surgical drill for osteotomy

(76) Inventor: Chih Chung Ho, Meinong Town (CN)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/242,579

(22) Filed: Oct. 3, 2005

(51) Int. Cl. A61B 17/00

(52) U.S. Cl. 606/80

(57) ABSTRACT

The invention relates to a surgical drill for osteotomy. The surgical drill comprises: a shank, a base portion, a cutting blade, a non-cutting section and a groove. The base portion is formed on the shank. The cutting blade is formed on the edge of the base portion. The non-cutting section is formed on the top of the base portion. The groove is formed on a peripheral section of the base portion, and is adjacent to the cutting blade and extends along a longitudinal direction. According to the invention, the surgical drill utilizes one single cutting blade to drill a socket formed by an implant drill. Because the fewer the blades are, the slower the working speed can be, so the bottom of the socket is not easy to penetrate the bony structure. By using the surgical drill of the invention, an intact bony plate between the implant and sinus membrane is more likely to obtain. Another type of the surgical drill of the invention can turn a tapered socket into a straight one at the posterior mandible.

### FIELD OF THE INVENTION

The present invention relates to a surgical drill, more particularly, a surgical drill for osteotomy.

### BACKGROUND OF THE INVENTION

When the thickness of the posterior maxilla is not enough for a dental implant, an internal sinus lift is recommended. By using the instrument named osteotome, the sinus-lifting surgery may be fulfilled. But the surgery is skill-emphasized. So a drill that makes the surgery of internal sinus lift easier and safer is helpftul.

Referring to FIG. 1, it shows a conventional scoring device. U.S. Patent Publication No. 2005/0064368, entitled “DENTAL IMPLANT SYSTEM AND METHOD,” discloses a scoring device 10 for scoring the alveolar bone at the top end of hole formed by an implant drill. The scoring device 10 comprises a scoring structure 11 extending upwardly from a top structure of the scoring device 10. The scoring structure 11 is typically a raised scoring edge extending around the circumference of a cavity 12. The scoring structure 11 is typically circular and has an outer diameter less than an outer diameter of the scoring device 10. The scoring device 10 further includes an exterior cutting surface 13 and longitudinal grooves 14. The implant drill is used to pre-drill a hole in alveolar bone of the maxilla.

The scoring device 10 is used to score the alveolar bone at the top end of the hole. Before an internal sinus-lifting surgery is to be practiced, the hole is prepared by the scoring device 10. The bottom of the hole is suggested not to penetrate the bony structure, and later the osteotome is inserted to breach the bony plate and finally push up the sinus membrane. However, the scoring structure 11 of the conventional scoring device 10 is not easy to manipulate, because it contains multiple cutting blades.

Besides, at the posterior mandible, the mandibular canal is usually the limit of the dental implant. Oftentimes, a short implant is the only choice. Because a tapered implant is less capable to resist the occlusal force than a straight implant, the latter is a better selection. Generally, the socket formed by the implant drills is tapered. Therefore, a drill that turns the tapered socket into a straight one to accommodate a straight implant is in demand.

Therefore, it is necessary to provide a surgical drill so as to solve the above problems.

### SUMMARY OF THE INVENTION

One objective of the present invention is to provide a surgical drill for osteotomy. The surgical drill comprises: a shank, a base portion, a cutting blade, a non-cutting section and a groove. The base portion is formed on the shank. The cutting blade is formed on the edge of the base portion. The non-cutting section is formed on the top of the base portion. The groove is formed on a peripheral section of the base portion, and is adjacent to the cutting blade and extends along a longitudinal direction.

According to the invention, the surgical drill utilizes one single cutting blade to drill a socket formed by an implant drill. Because the fewer the blades are, the slower the working speed can be, so the bottom of the socket is not easy to penetrate the bony structure. By using the surgical drill of the invention, an intact bony plate between the implant and sinus membrane is more likely to obtain. Another type of the surgical drill of the invention can turn a tapered socket into a straight one at the posterior mandible.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a conventional scoring device having a plurality of cutting blades;

FIG. 2 shows a perspective view of the surgical drill, according to a first embodiment of the invention;

FIG. 3 shows a perspective view of the surgical drill, according to a second embodiment of the invention;

FIG. 4 shows a front view of the surgical drill, according to the first embodiment of the invention;

FIG. 5 shows a front view of the surgical drill, according to a third embodiment of the invention;

FIG. 6 shows a front view of the surgical drill, according to a fourth embodiment of the invention;

FIG. 7A illustrates a socket prepared by implant drills;

FIG. 7B illustrates that the surgical drill is utilized to further drill the socket, according to the first embodiment of the invention;

FIG. 7C illustrates the socket after the surgical drill is utilized to further drill the socket, according to the first embodiment of the invention;

FIG. 8A illustrates a socket prepared by implant drills;

FIG. 8B illustrates that the surgical drill is utilized to further drill the socket, according to the second embodiment of the invention; and

FIG. 8C illustrates the socket after the surgical drill is utilized to further drill the socket, according to the second embodiment of the invention

### DETAILED DESCRIPTION OF THE PREFERRED EMBODIMENT

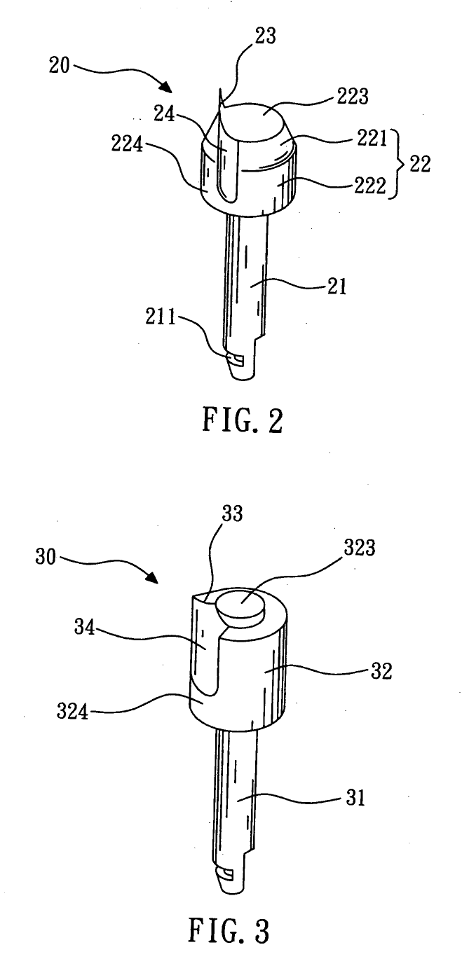

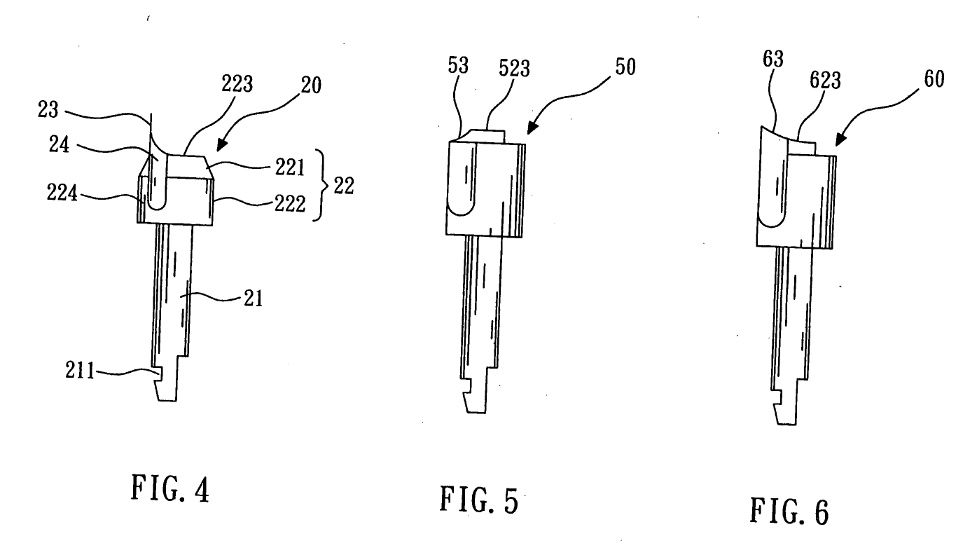

Referring to FIGURES. 2 and 4, according to a first embodiment of the invention, the surgical drill 20 comprises: a shank 21, a base portion 22, a cutting blade 23 and a groove 24. The shank 21 comprises a clip 211 for mounting mechanically driven rotation.

The base portion 22 is formed on the shank 21. The base portion 22 comprises a first section 221 and a second section 222. The first section 221 is disposed on the second section 222. The first section 221 has a first diameter D1, and the second section 222 has a second diameter D2. The first diameter D1 is smaller than or equal to the second diameter D2. Typically, the base portion 22 is cylindrical shape. The base portion 22 has a non-cutting section 223 and a peripheral section 224. The non-cutting section 223 is a plane section and is disposed on the top of the base portion 22.

The cutting blade 23 is formed on the edge of the base portion 22. The non-cutting section 223 is formed on the top of the base portion 22. The cutting blade 23 extends upwardly from the base portion 22, and is formed as a sharp shape. Besides, in the first embodiment, the cutting blade 23 is formed on the first section 221 of the base portion 22. The length of the cutting blade 23 is suggested to be about 1-2 mm.

The groove 24 is formed on the peripheral section 224 of the base portion 22, and is adjacent to the cutting blade 23 and extends along a longitudinal direction. The groove 24 is used to allow flow of blood and bony material cut by the cutting blade 23 along the peripheral section 224 of the base portion 22.

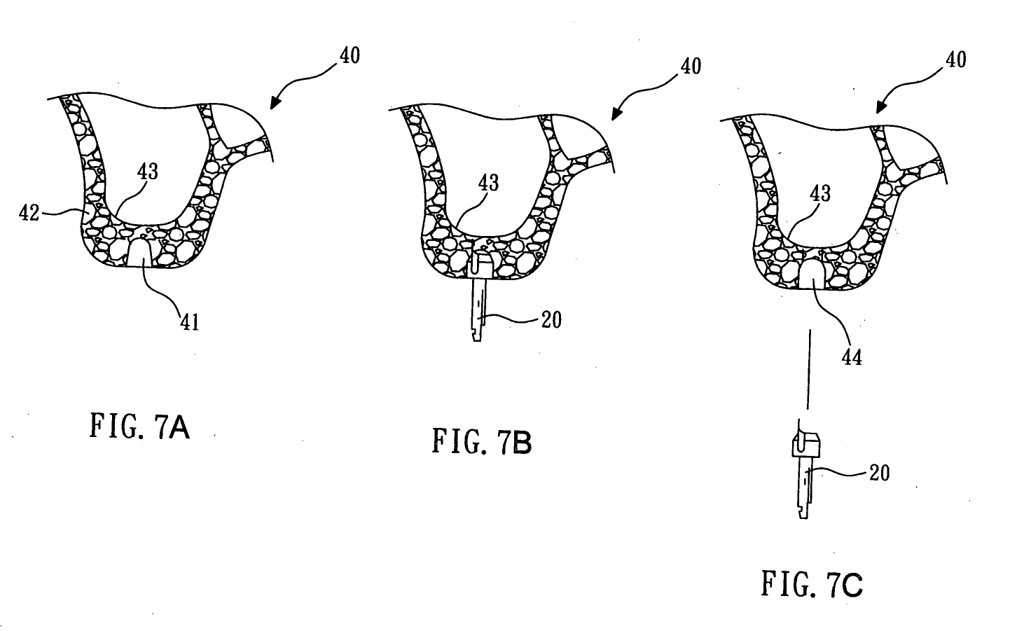

Referring to FIGURES. 7A to 7C, they illustrates that the surgical drill 20 is utilized to further drill the socket, according to the first embodiment of the invention. The surgical drill 20 of the present invention is suggested to be used before the manipulation of the osteotome. When an internal sinus-lifting surgery is to be practiced, a socket 41 is prepared by the implant drills. The bottom of the socket 41 is suggested not to penetrate the bony structure 42, and later the osteotome is inserted to breach the bony plate and finally push up the sinus membrane 43. Clinically, an intact bony plate between the implant and the sinus membrane 43 is preferred. But the implant drill may prepare the bony socket 41 with a concave bottom, as shown in FIG. 7A. So the osteotome technique for internal sinus lift cannot always get an intact bony plate between the implant and the sinus membrane. The socket 41 is suggested to be prepared with the bottom 2-3 mm from the sinus floor.

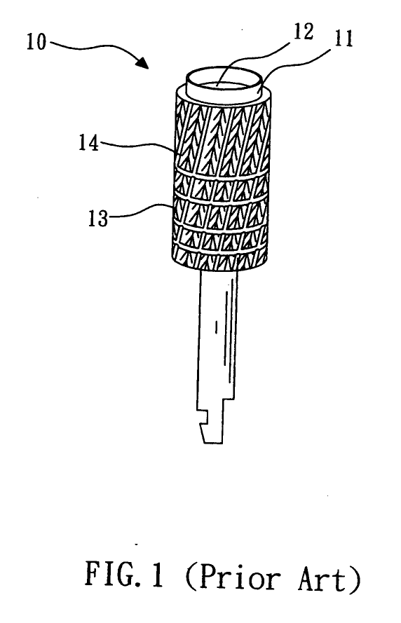

Referring to FIG. 7B, the surgical drill 20 is utilized to further drill the socket 41. The surgical drill 20 of the invention is inserted to work in. With one single cutting blade 23, the working speed can go slower than that with scoring structure 11 of the conventional scoring device 10 in FIG. 1. The slower the speed is, the safer the preparation of the socket 41 can be. After the function of the cutting blade 23 is stopped by the non-cutting section 223, the surgical drill 20 is retreated from the socket 44, as shown in FIG. 7C.

Then, an osteotome that is compatible to the preparation is used to push up the plate and elevates the sinus membrane 43. According to the surgical drill of the invention, an intact bony plate between the implant and sinus membrane 43 is more likely to obtain. The working depth of the surgical drill is doubly checked if the base portion is not a straight cylinder. That is, the base portion 22 comprises a first section 221 and a second section 222, and the first diameter D1 of the first section is smaller than the second diameter D2 of the second section 222. Because the base portion 22 is not a straight cylinder, the working depth of the surgical drill 20 can be doubly checked.

Slow working speed would lead to less damage if the plate of the socket bottom is accidentally over prepared. Because the surgical drill 20 utilizes one single cutting blade 23 to drill the socket 41. The slower speed would make the surgeons easier to notice the penetration.

Referring to FIG. 3, according to a second embodiment of the invention, the surgical drill 30 comprises: a shank 31, a base portion 32, a cutting blade 33 and a groove 34. The base portion 32 is formed on the shank 31, and the base portion 32 is substantially a cylinder. The base portion 32 has a non-cutting section 323 and a peripheral section 324. The non-cutting section 323 is a plane section and is disposed on the top of the base portion 32.

The cutting blade 33 is formed on the base portion 32, and extends from an edge of the base portion 32 to the non-cutting section 323 of the base portion 32. The cutting blade 33 extends transversely from the edge of the base portion to the non-cutting section 323 of the base portion 32. The non-cutting section 323 is a plane section and is formed on the central top region of the base portion 32. The non-cutting section 323 occupies about ½ of the base portion. In the third embodiment of the invention, the cutting blade 33 and the non-cutting section 323 are at the same level. The groove 34 is formed on the peripheral section 324 of the base portion 32, and is adjacent to the cutting blade 33 and extends along a longitudinal direction.

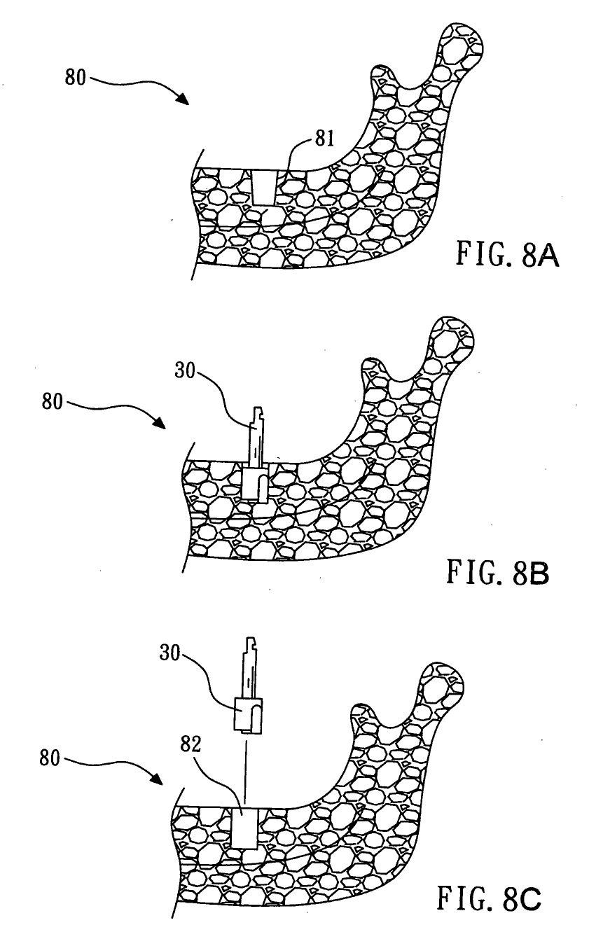

Referring to FIGS. 8A to 8C, they illustrates that the surgical drill 30 is utilized to further drill the socket, according to the second embodiment of the invention. The surgical drill 30 of the invention is generally used to turn a tapered socket into a straight one at the posterior mandible 80. The socket 81 formed by implant drills is tapered as shown in FIG. 8A. Because the base portion 32 is a straight cylinder, the surgical drill 30 can be used to further drill the tapered socket 81 into a straight socket 82 as shown in FIGS. 8B and 8C.

Referring to FIG. 5, it shows a surgical drill, according to a third embodiment of the invention. Compared with the surgical drill 30 of the second embodiment of the invention, the cutting blade 53 of the surgical drill 50 is lower than the non-cutting section 523. Referring to FIG. 6, it shows a surgical drill according to a fourth embodiment of the invention. The cutting blade 63 of the surgical drill 60 is higher than the non-cutting section 623. The surgical drill 50 of the third embodiment and the surgical drill 60 of the fourth embodiment can be used to meet the clinical situation.

While the embodiments of the present invention have been illustrated and described, various modifications and improvements can be made by those skilled in the art. The embodiments of the present invention are therefore described in an illustrative, but not restrictive, sense. It is intended that the present invention may not be limited to the particular forms as illustrated, and that all modifications which maintain the spirit and scope of the present invention are within the scope as defined in the appended claims.

The embodiments of the invention in which an exclusive property or privilege is: claimed are defined as follows:

1. A surgical drill for osteotomy, comprising:
a shank;
a base portion, formed on the shank;
a cutting blade, formed on the edge of the base portion;
a non-cutting section, formed on the top of the base portion; and
a groove, formed on a peripheral section of the base portion, being adjacent to the cutting blade and extending along a longitudinal direction.

2. The surgical drill according to claim 1, wherein the cutting blade extends upwardly from the base portion.

3. The surgical drill according to claim 2, wherein the base portion comprises a first section and a second section, the first section is disposed on the second section, the first section has a first diameter, the second section has a second diameter, the first diameter is smaller than or equal to the second diameter, and the cutting blade is formed on the first section.

4. The surgical drill according to claim 1, wherein the cutting blade extends transversely from the edge of the base portion to the non-cutting section of the base portion.

5. The surgical drill according to claim 4, wherein the non-cutting section is a plane section and is formed on the central top region of the base portion.

6. The surgical drill according to claim 4, wherein the cutting blade and the non-cutting section are at the same level.

7. The surgical drill according to claim 4, wherein the cutting blade is lower than the non-cutting section.

8. The surgical drill according to claim 4, wherein the cutting blade is higher than the non-cutting section.
